(19) United States
(12) Patent Application Publication
(10) Pub. No.: US 2015/0272653 A1
(43) Pub. Date: Oct. 1, 2015

(54) Thermal therapy ablation detection with ultrasound

(71) Applicant: Siemens Medical Solutions USA, Inc., Malvern, PA (US)

(72) Inventors: Shelby Brunke, Sammamish, WA (US); Stephen Hsu, Issaquah, WA (US)

(21) Appl. No.: 14/567,946

(22) Filed: Dec. 11, 2014

Related U.S. Application Data
(60) Provisional application No. 61/973,668, filed on Apr. 1, 2014.

(51) Int. Cl. A61B 18/04; A61B 19/00

(52) U.S. Cl. CPC A61B 18/04; A61B 19/5225; A61B 2018/00577

(57) ABSTRACT

Thermal therapy ablation detection uses medical diagnostic ultrasound. Since acoustically measured information becomes unreliable for temperature estimation at a temperature close the time at which treatment is complete, the information is instead or additionally used to detect a tissue condition indicating sufficient treatment, such as detecting cell death. Using multiple different types of parameters as input and/or a machine-learnt classifier, the completion of treatment from a tissue alteration perspective is detected using the transition that makes temperature estimation less reliable.

### RELATED APPLICATIONS

The present patent document claims the benefit of the filing date under 35 U.S.C. §119(e) of Provisional U.S. Patent Application Ser. No. 61/973,668, filed Apr. 1, 2014, which is hereby incorporated by reference.

### BACKGROUND

The present invention relates to thermal therapy with ultrasound monitoring. Thermal energy-based treatments apply heat within a patient. Various modalities, such as RF ablation, microwave, laser irradiation, or high intensity focused ultrasound (HIFU), deliver energy. The safety and efficacy of these treatments are closely correlated with both the end-of-dose tissue temperatures and the time-temperature history of the treated tissue. Time-temperature history is quantified as “thermal dose.”

The temperature and dose are monitored using invasive sensors, such as needle probes. Invasive procedures may be undesired. Magnetic resonance imaging (MRI) monitoring noninvasively measure tissues treatment temperatures. MRI approaches may not provide real-time feedback and/or are expensive. Ultrasound may be used for non-invasive monitoring. US Published Patent Application 2011/0060221 teaches estimation of tissue temperature using a neural network. Acoustic information derived from imaging modes is input to a neural network. The neural network estimates the temperature based on the acoustic information, so may estimate in real time. When the absolute temperature of the tissue reaches approximately 55-57 degrees C. through the delivery of high intensity focused ultrasound, the acoustic signals input to the neural network estimator go through a rapid change in character. The acoustic signals deteriorate to the point that accurate temperature measurement is confounded.

### BRIEF SUMMARY

By way of introduction, the preferred embodiments described below include methods, computer readable media, instructions, and systems for thermal therapy ablation detection with medical diagnostic ultrasound. Since the acoustically measured information becomes unreliable for temperature estimation at a temperature close the time at which treatment is complete, the information is instead or additionally used to detect a tissue condition indicating sufficient treatment, such as detecting cell death. Using multiple different types of parameters as input and/or a machine-learnt classifier, the completion of treatment from a tissue alteration perspective is detected using the transition that makes temperature estimation less reliable.

In a first aspect, a method of thermal therapy ablation detection with medical diagnostic ultrasound is provided. An ultrasound system acquires ultrasound data from a scan of tissue of a patient undergoing thermal therapy. A processor derives information from the ultrasound data. The processor detects, by applying a classifier, a time point of death of the tissue based on an output of the classifier in response to input of the information. An indication of the time point is output.

In a second aspect, a non-transitory computer readable storage medium has stored therein data representing instructions executable by a programmed processor for thermal therapy ablation detection with medical diagnostic ultrasound. The storage medium includes instructions for: scanning, with a transducer, a patient with ultrasound during the thermal therapy; calculating, with an ultrasound scanner, first and second types of tissue characteristics over time from response to the scanning; identifying, by the processor and from the first and second types of tissue characteristics, a transition associated with denaturation of tissue; and indicating the transition.

In a third aspect, a system is provided for thermal therapy ablation detection with medical diagnostic ultrasound. A receive beamformer is configured to acquire ultrasound data representing a region of a patient. A processor is configured to determine cell death in the region with a machine-trained classifier and an input feature vector of the machine-trained classifier comprising two or more types of parameters derived from the ultrasound data. A display is configured to display an indication of the cell death.

Further aspects and advantages of the invention are discussed below in conjunction with the preferred embodiments. The present invention is defined by the following claims, and nothing in this section should be taken as a limitation on those claims.

### BRIEF DESCRIPTION OF THE DRAWINGS

The components and the figures are not necessarily to scale, emphasis instead being placed upon illustrating the principles of the invention. Moreover, in the Figures, like reference numerals designate corresponding parts throughout the different views.

FIG. 1 is a flow chart diagram of one embodiment of a method for thermal therapy ablation detection with medical diagnostic ultrasound;

FIG. 2 is an example image of tissue showing a region of cell death in thermal therapy;

FIG. 3 is a graph illustrating a deviation between a measured temperature and an estimate temperature;

FIG. 4 is a block diagram of one embodiment of a system for thermal therapy ablation detection with medical diagnostic ultrasound.

### DETAILED DESCRIPTION OF THE DRAWINGS AND PRESENTLY PREFERRED EMBODIMENTS

Acoustic signals, as derived from multiple imaging modes (e.g. strain, displacement, backscattered power, etc. . . . ), are monitored through the course of therapy. The temperature of the tissue around a therapeutic target may be monitored using a recurrent neural network applied to these signals. At a repeatable absolute temperature, the acoustic signals input to the classifier sharply change. This repeatable or reproducible change in the signal characteristics is used as an acoustic signature of tissue changes associated with cell death.

Acoustic thermometry is a relative (as opposed to absolute) temperature estimation scheme and is known to be more accurate at low delta-T's (up to about 15-20 deg. C. delta T). Using neural network-based technology, this range is increased. However, the error in the temperature and therefore thermal dose increases with delta T. This means that the precise thermal dose as a time-temp integral is prone to error. Machine learning techniques are accurate at detecting a threshold change in multiple signals associated with the tissue changes that accompany a lethal thermal dose (protein denaturation, etc. . . . ).

In one embodiment, a combination that meets the clinical need and combines the strengths of the two technical approaches is used. Acoustic thermometry is provided for low delta T monitoring of tissue changes combined with a machine learning approach to detecting a lethal dose based on multiple acoustic signal inputs. A machine learnt classifier (e.g., neural network) takes a multitude of signals as inputs and outputs a relative temperature estimate, and another machine learnt classifier detects the transition of tissue from viable to thermally ablated using a multitude of signals as inputs. The endpoint might be a hard binary threshold (cooked, not cooked), or a softer threshold as a % complete, for example. The two technologies are combined in a manner that supports complete thermal therapy monitoring from beginning of therapy through lethal dose. The application visualizes the tissue temperatures within the region or volume of interest along with a visualization of ablation regions.

The temperature estimate and ablation detection data may be processed and displayed in many formats. In addition to direct imaging visualization, alerts and warning may be issued given doses relative to intended targets and critical structures. Therapy control may be automated based on outputs from the ablation detection.

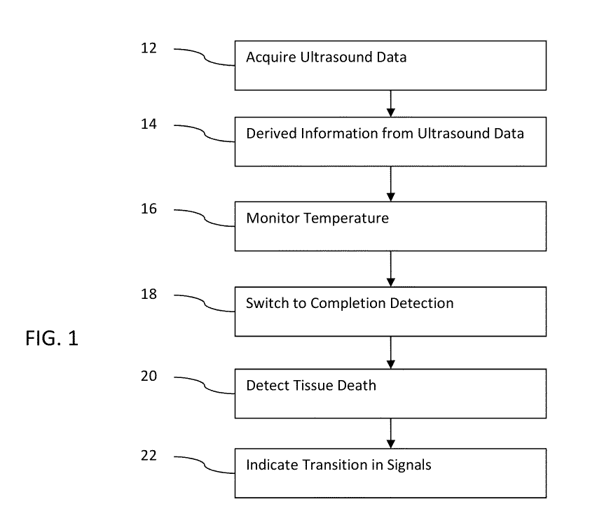

FIG. 1 shows one embodiment of a method of thermal therapy endpoint or ablation detection with medical diagnostic ultrasound. This embodiment is directed to monitoring temperature and then triggering detection of the endpoint at which cell death occurs. The ablation of the tissue is detected. In other embodiments, the detection of the endpoint is performed without monitoring temperature (e.g., without acts 16 and 18).

Additional, different, or fewer acts may be provided. For example, the percent ablation, trend, or closeness to cell death is detected in act 20 instead of detecting cell death or the endpoint of ablation.

The temperature monitoring of act 16 and/or the detection of tissue death of act 20 are performed with machine learnt classifiers. Separate classifiers are provided. In other embodiments, one classifier is trained for both outputs. In yet other embodiments, acts for training the classifier are provided with or without acts for applying the learnt classifier.

The acts are performed in the order shown or a different order. The acts are performed during therapy. The acts are repeated throughout the therapy. For example, a reference set of data is acquired before application of the therapy. One or more parameters may be assumed for the initial iteration, such as assuming a temperature common for patients or type of tissue within a patient. Once thermal therapy begins, the acts are repeated to provide updated measurements and resulting predictions, estimations, or detection. Changes in parameters may be used as input features with or without other parameters. A time history of the input parameters, current values, change in values, or other derived information may be used for monitoring temperature and/or detecting tissue death. The current estimated temperature, dose, and/or detection of tissue death may be used to determine whether, where, and/or at what level to continue the therapy. In other embodiments, the endpoint showing tissue death is determined during later review.

In act 12, ultrasound data from a scan of tissue of a patient undergoing thermal therapy is acquired. The ultrasound data represents the patient. A medical diagnostic ultrasound system applies electrical signals to a transducer, which then converts the electrical energy to acoustic energy for scanning a region of the patient. Echoes are received and converted into electrical signals by elements of the transducer for receive operation of the scan. Any type of scan, scan format, or imaging mode may be used. For example, harmonic imaging is used with or without added contrast agents. As another example, B-mode, color flow mode, spectral Doppler mode, M-mode, or other imaging mode is used.

Ultrasound data representing anatomical information is acquired from a patient. The ultrasound data represents a point, a line, an area, or a volume of the patient. Waveforms at ultrasound frequencies are transmitted, and echoes are received. The acoustic echoes are converted into electrical signals and beamformed to represent sampled locations within a region of the patient. The beamformed data may be filtered or otherwise processed. The beamformed data may be detected, such as determining an intensity (e.g., B-mode or backscatter power or intensity). A sequence of echo signals from a same location may be used to estimate velocity, variance, and/or energy. Echoes at one or more harmonics of the transmitted waveforms may be processed. The detected values may be filtered and/or scan converted to a display format. The ultrasound data representing the patient is from any point along the ultrasound processing path, such as channel data prior to beamformation, radio frequency or in-phase and quadrature data after beamforming but prior to detection, detected data, or scan converted data.

Ultrasound data may be pre-detected data or may be detected data. For example, B-mode data represents tissue structures. As another example, flow data indicates locations associated with a vessel or flow. Alternatively or additionally, the ultrasound data is derived from detected data. For example, a type of tissue at a given location is determined from a speckle characteristic, echo intensity, template matching with tissue structure, or other processing. As another example, region growing is used with B-mode data or color flow data to determine that the ultrasound data represents a vessel or other fluid region. A current distribution of anatomy, such as a list of represented organs, may be determined. The actual data and/or derived information are parameters to be used in combination with the classifier.

In act 14, ablation related measurements are performed. A processor derives information from the ultrasound data. Using channel, beamformed, and/or detected ultrasound data, the processor derives information for input to the classifier. The ultrasound scanner, with a transducer, is used to acquire some or all of the ultrasound data then used to derive inputs. The ultrasound data itself may be used as information for input.

Information may be derived from the ultrasound data. Any tissue characteristic related measurement may be used. For example, tissue becomes less elastic upon cell death. Measuring the elasticity may indicate cell death. Temperature related measurements may directly or indirectly indicate a temperature corresponding to cell death, such as 57 degrees C. The measurements may be for raw ultrasound data or may be derived from ultrasound data.

Only one, or two or more measurements are performed. Measurements are performed for just one location or for each of multiple locations in a region. Full or sparse sampling may be used. The measurements are performed over time, but independent of previous measurements. Alternatively or additionally, a change in a measurement from a reference or any previous (e.g., most recent) measurement may be used.

In one embodiment, two or more ultrasound measurements are performed with or without other tissue state-related measurements. Different types of information are derived. Ultrasound measurements may be provided for a plurality of different locations in and/or around the treatment region. Any now known or later developed measurement using ultrasound may be used. In one embodiment, two or more, such as all four, of tissue displacement, speed of sound, backscatter intensity, and a normalized correlation coefficient of received signals are performed. Other measurements are possible, such as expansion of vessel walls. Strain or other elasticity measurements may be derived from the ultrasound data.

Tissue displacement is measured by determining an offset in one, two, or three-dimensions. A displacement associated with a minimum sum of absolute differences or highest correlation is determined. The current scan data is translated, rotated, and/or scaled relative to a reference dataset, such as a previous or initial scan. The offset associated with a greatest or sufficient similarity is determined as the displacement. B-mode or harmonic mode data is used, but other ultrasound data may be used. The displacement calculated for one location may be used to refine the search or search region in another location. Other measures of displacement may be used.

The speed of sound may be measured by comparison in receive time from prior to heating with receive time during heating. A pulse is transmitted. The time for the echo to return from a given location may be used to determine the speed of sound from the transducer to the location and back. Any aperture may be used, such as separately measuring for the same locations with different apertures and averaging. In another embodiment, signals are correlated. For example, in-phase and quadrature signals after beamformation are correlated with reference signals. A phase offset between the reference and current signals is determined. The frequency of the transmitted waveform (i.e., ultrasound frequency) is used to convert the phase difference to a time or speed of sound. Other measurements of the speed of sound may be used.

The backscatter intensity is B-mode or M-mode. The intensity or energy of the envelope of the echo signal is determined.

The normalized correlation coefficient of received signals may be measured. Beamformed data prior to detection, such as in-phase and quadrature data, is cross-correlated. In one embodiment, a reference sample or samples are acquired. During treatment, subsequent samples are acquired. For each location, a spatial window, such as three wavelengths in depth, defines the data for correlation. The window defines a length, area or volume. The current data is correlated with the reference data within the window space. The normalized cross-correlation is performed for the data in the window. As new data is acquired, further cross-correlation is performed. The correlation indicates an amount of decorrelation. The measure of correlation or decorrelation may be derived.

Any tissue-state associated acoustic and physical parameters or changes in the parameters may be measured. Combinations of parameters may be used as the input information. Other measurements include tissue elasticity, thermal strain, strain, strain rate, motion (e.g., displacement or color flow measurement), shear wave velocity, shear modulus, viscosity, ultrasonic spectrum characteristic, or reflected power (e.g., backscatter cross-section).

The information derived reflects the effect of therapy on tissue. The effect may be related to instantaneous temperature or may be the result of application of heat beyond a particular dosage. The effect may persist after removal of the heat. Therapeutic effect- and bio effect-associated parameters include elasticity (e.g., acoustic radiation force imaging), expansion (e.g., determined from B-mode tracking), shrinkage (e.g., determined from B-mode tracking), phase change, water content, flow or other fluid changes (e.g., coagulation determined from Doppler information), and/or other measurable changes.

Other therapy data may be received or derived for use as an input to the classifier. The intensity or characteristics (e.g., applied dose) of the therapy may be used. Changes in the therapy data parameters or history may be used.

Clinical or other information may be acquired. For example, genetic information or other tissue related data may be mined from a patient record. Any feature contributing to determination of information reflecting effect on tissue may be used.

The derived information may use non-ultrasound modalities. For example, a thermocouple, infrared, or other sensor is used. The sensor is inserted within the patient or scans the patient. As another example, information from the therapeutic treatment device is used. An energy output, dose, or other parameter of the thermal treatment is measured or received.

Non-real time measurements may be used, such as a baseline temperature. MRI-based measurements for temperature distribution in a region may be used. Real-time measurements may be used, such as associated with ultrasound measurements performed during application of thermal therapy to a region of the patient.

The information is used as inputs to a model or to calculate values for input to the model. The derived information is provided for one or more locations, such as deriving from ultrasound data for all locations in a two- or three-dimensional region. Alternatively, the derived information is generally associated with the entire region, such as one dose or energy level for the entire region.

In optional act 16, the temperature at one or more locations is monitored. The monitoring is during the thermal therapy. As heat is applied or generated in the tissue of the patient, the temperature at the focus of the heat or in a two- or three-dimensional region around the focus is monitored.

Any invasive or non-invasive temperature monitoring may be used. In one embodiment, the temperature is monitored using the response to the scanning. The ultrasound data, derived information, other ultrasound data, other derived information, or combinations thereof are used to monitor temperature. For example, the temperature is monitored using the machine learnt neural network or other estimator disclosed in US Published Patent Application 2011/0060221, the disclosure of which is incorporated herein by reference. A classifier trained for estimating temperatures at many locations outputs the estimates of temperature over time during the therapy.

Two stages of therapy monitoring are provided. In the first stage of therapy application, from therapy energy onset to imminent cell death, acoustic thermometry is used as an estimator to estimate and output an image of thermal energy distribution. These spatial temperature estimates ensure that energy for therapy is properly focused and critical structures are not inadvertently heated. The user may make adjustments based on the temperature estimates and/or the processor may cause the therapy device to automatically adjust the focus, energy magnitude, distribution of energy in time or space, or other therapy characteristic based on feedback of the estimated temperature distribution.

In the second stage of therapy application, a detector detects changes in acoustic data and/or derived information indicative of cell death in the underlying tissue. Since the temperature estimate become less reliable at higher temperatures (e.g., 55 degrees C. or higher) and the desired temperature for treatment may be greater (e.g., 57 degrees C. or higher), the second stage is implemented to inform the user or the therapy system when cell death is estimated to be occurring at one or more locations after temperature estimates become less reliable. Alternatively or additionally, the ablation may be detected as a prediction or amount of doneness (e.g., 80% ablation indicating cell death at 100%).

In act 18, the monitoring of act 16 switches to the detection of act 20. Once the temperature at one or more locations reaches a certain point, the flow changes to identifying cell death or % of cell death rather than temperature. In alternative embodiments, both temperature estimation and detection of cell death are performed from the beginning of therapy application. In other alternatives, the detection of cell death is triggered by the temperature estimates, but the temperature estimation continues without stopping once the detecting operation starts. The estimator of temperature and detector of cell death algorithms or classifiers may be based on the same underlying structure (e.g., train one classifier to detect both) or may use separate trained classifiers using the same or different underlying machine learning approaches. The detection of cell death may rely on many of the same inputs as the estimator of temperature. Since the temperature inputs consistently transition at the appropriate points in therapy, these inputs may be used to detect cell death. The detector algorithm may be a standalone system for determining the successful therapeutic ablation.

Any temperature may be used for switching. For example, the temperature reaching 50, 51, 52, 53, 54, or 55 at a location of maximum temperature or focus location is used. As another example, an average of the highest X number of locations is used, such as an average temperature for the 10 hottest locations being above the threshold. In one embodiment, the threshold is set based on imminent cell death. Cell death typically occurs around 57 degree C. for an average dose (magnitude over time). The threshold is set one or a few degrees less. Where the rate of energy application is different, different thresholds may be used.

In act 20, a time point of death of tissue is detected. A processor applies a classifier. An input feature vector of information is input to the classifier. The processor applies a matrix or other classifier construction to output the time point of death of the tissue. Alternatively, a prediction or % of cell death may be detected.

In one embodiment, the classifier is a machine trained neural network. By the processor applying the machine learnt neural network, the time point or occurrence of the cell death for one or more locations is output. A neural network or other method of artificial intelligence is employed to detect the transition in the state of the tissue based on the transition, change, or state of the input information.

Any of various classifiers may be used. Any model may be used, such as a neural network or a piecewise linear model. The model is programmed or designed based on theory or experimentation. In one embodiment, the model is a machine-learned model. The model is trained from a set of training data labeled with a ground truth, such as training data associated with actual tissue state over time or tissue states at given times. For example, the various information or receive data are acquired over time for each of multiple patients. During thermal therapy, the state of the tissue is determined by an expert. The state of the tissue or whether the tissue is dead or not is the ground truth. Through one or more various machine-learning processes, the classifier is trained to detect cell death given the values and/or any feedback.

Any machine-learning algorithm or approach to classification may be used. For example, a support vector machine (e.g., 2-norm SVM), linear regression, boosting network, probabilistic boosting tree, linear discriminant analysis, relevance vector machine, neural network, combinations thereof, or other now known or later developed machine learning is provided. The machine learning provides a matrix or other output. The matrix is derived from analysis of a database of training data with known results. The machine-learning algorithm determines the relationship of different inputs to the result. The learning may select only a sub-set of input features or may use all available input features. A programmer may influence or control which input features to use or other performance of the training. For example, the programmer may limit the available features to information available in real-time. The matrix associates input features with outcomes, providing a model for classifying. Machine training provides relationships using one or more input variables with outcome, allowing for verification or creation of interrelationships not easily performed manually.

The model represents a probability of tissue death-related information. This probability is a likelihood for the tissue being dead. A range of probabilities associated with different possible tissue states (e.g., binary dead or not dead or a three or more possible states) is output. Alternatively, the tissue state with the highest probability is output. In other embodiments, the tissue state or binary dead or not dead information is output without probability information.

As an alternative to machine learning, a manually programmed classifier may be used. The classifier may be validated using machine training or other processes.

For application for a specific patient, the detection is based input of the information. The time point of cell death or amount of progression to cell death is detected from one or more types of information. By using different types of information, a more accurate classification may be provided. Any ultrasound data, information derived from ultrasound data, and/or non-ultrasound information may be included in the input vector. For example, the time point is detected in response to strain, the signal decorrelation, and B-mode data (e.g., backscatter intensity). The different information represents different characteristics, such as different elasticity characteristics. As another example, acoustic signals and information derived from the acoustic signals used as input to the detection algorithm include strain, displacement, backscattered power, signal decorrelation, shear wave velocity, any other metric of tissue elasticity, or other information.

The tissue state related information and/or the therapy data are applied to the classifier. The information or data are input as raw data. Alternatively, the values (i.e., measurements and/or data) are processed and the processed values are input. For example, the values are filtered spatially and/or temporally. As another example, a different type of value may be calculated from the values, such as determining a variance, a derivative, normalized, or other function from the values. In another example, the change between the current values and reference or previous values is determined. A time-history of the values over a window of time may be used. The values are input as features of the classifier.

The output of the classifier may be used as an input. The values are applied during the application of thermal therapy. For an initial application of the classifier, the feedback is replaced with a reference tissue state, such as tissue at a start or initial state (i.e., healthy or cancerous). For further application of the classifier, the previous output is fed back as an input, providing a time-dependent classifier. The tissue state information output by the classifier is fed back as a time history of the information, such as state of tissue at one or more other times. During thermal therapy, the measured or received values are updated (i.e., current values are input for each application of the classifier), but previous values may also be used. The feedback provides an estimated spatial distribution of tissue state or related information in the region at a previous time. The subsequent output of the classifier is a function of the ultrasound data or other values and a previous output of the detecting. The time-history of the values may be used as inputs, such that the time history and spatial distributions of the tissue state (e.g., therapeutic effect-related parameters) are used as features of the classifier.

The information and/or data input for application of the classifier represents a distinct time. The values for the tissue at a time are input. The classifier detects based on the values at the time. The classifier is periodically applied to determine the state of the tissue at that time. Alternatively, the classifier uses a change, trend, or other information derived from the values over time. The classifier is periodically applied to determine the state of the tissue at that time but using values for the time and other times.

The classifier detects the time point of death of the tissue. The time of denaturation or other change in tissue state is detected. The thermal therapy, after a certain dose or in response to a given temperature, causes cell death, killing the tissue. Since the therapy is not isolated to a point, the state of the tissue is detected for more than one location. The state of the tissues over a one, two, or three-dimensional distribution of locations in the patient are classified. For each location, the transition from living to dead tissue is detected as the endpoint. The therapy may continue, but the death of the tissue indicates that the therapy does not need to continue for that location. Detection of approaching or amount of ablation may be provided.

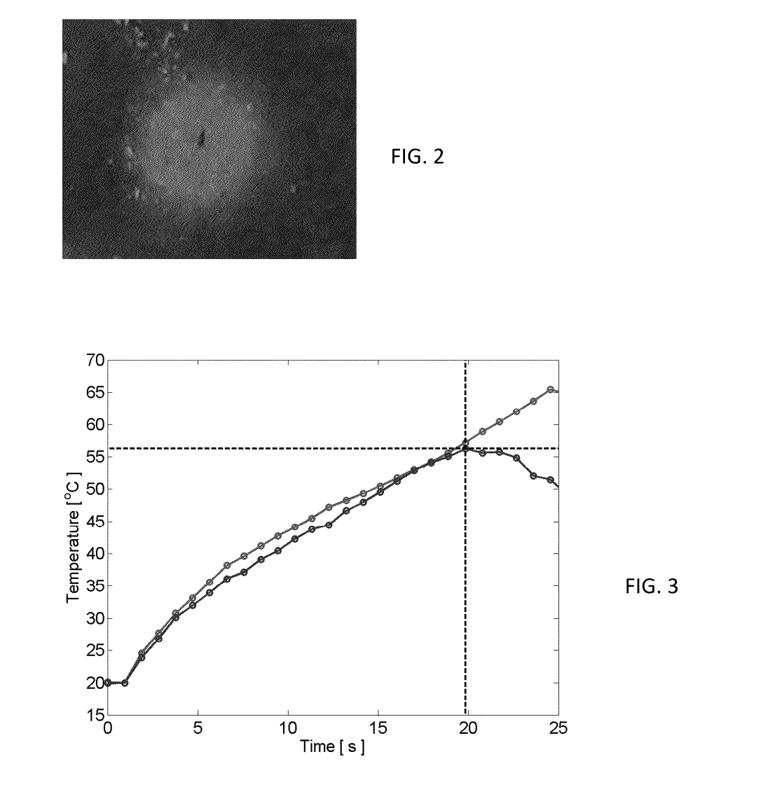

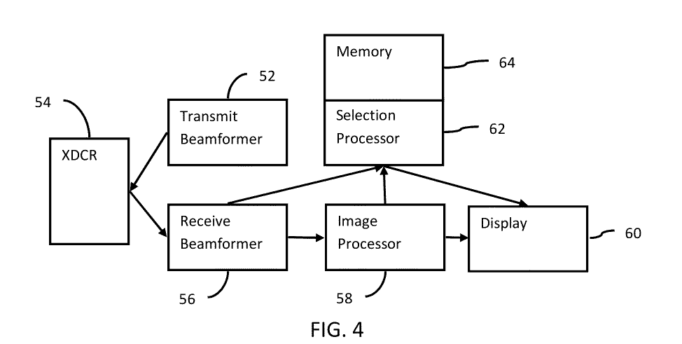

The classifier detects a signature pattern or one of several signature patterns of the input feature vector representing cell death of tissue. The transition in the input information making temperature measures less reliable coincides with changes in the (ex-vivo) tissue that are observed after cessation of therapeutic power delivery. FIG. 2 shows an image of ex-vivo bovine liver tissue used in a therapy experiment. The tissue is sectioned through the center of the high intensity focused ultrasound (HIFU) focus. The desiccated area in the center of the image corresponds to the HIFU focus. This tissue has essentially been “cooked” by the application of focused acoustic power. The observable change in inputs coincides with protein denaturation and desiccation. This therapeutic endpoint is detected from the repeatable and predictable change in the underlying acoustic signal or derived information as the signature of tissue changes associated with cell death.

Used in this manner, the neural network or other classifier is a detector of a physical state transition in the tissue. This transition is verified to be associated with cell death. For example, through monitoring the 240 equivalent minutes at 43 deg. C. and/or through histopathology verified studies, the verification is used as a ground truth for training. FIG. 3 illustrates the temperature estimate that changes slope, the actual thermocouple verified temperature that is linear throughout, the point of therapeutic (fatal) dose (57 degrees C.), and the point at which the acoustic signal undergoes transition so that the temperature classification deviates from the actual temperature (also 57 degrees C.). The signature that leads to the inaccuracy in temperature may be used to detect the state of the tissue.

In response to input of the features, the classifier outputs the tissue state. For example, the classifier uses displacement in two dimensions, elasticity in two dimensions, normalized cross-correlation coefficient in two dimensions, and backscatter intensity in two dimensions as input features. The classifier determines tissue state for locations distributed in two dimensions. The classifier outputs a tissue state or tissue state distribution (i.e., tissue state at different locations and/or times) from the input information. The resolution of the tissue state may be at any level, such as binary (dead or not dead). Alternatively, other tissue state-related information is output, such as a change in state.

In act 22, the transition to cell death is indicated. The time point of cell death is output. Any output may be used. In one embodiment, an image is output. The image shows distribution of tissue state. The tissue state may be displayed with other information, such as estimated temperature. For example, the color shows temperature and brightness or a different color is used to show dead tissue or tissue state. As another example, the tissue state is provided as an overlay on an ultrasound image representing the anatomy, such as overlaid on a B-mode image.

In other embodiments, the indication is an alert to the user in the form of a text display, audible sound, or other output. A graph of tissue state as a function of time or along a line may be displayed. A chart of probability of different states at different times may be output.

The indication is provided in real-time or as the transition is detected. The output is of the time point of tissue death. Alternatively, the indication is provided any amount of time after detection or predicted before detection. The output indicates the time point of tissue death, but may not be provided at that time.

In an alternative or additional embodiment, the time point is used to control the therapy. The control is manual, such as the user selecting adjustments or an endpoint for thermal therapy based on the tissue state information. Alternatively, the control is automatic, such as ceasing or varying therapy (e.g., magnitude, focus, or location of therapy) when the tissue state is reached at one or more locations. The dosing may be gradually reduced or increased as cell death and tissue denaturation is approached based on the detection of cell death and/or the temperature prior to cell death. In other embodiments, the tissue state from during the therapy or at an end of therapy is used to determine a prognosis or therapy result at a later time.

FIG. 5 shows one embodiment of a system for thermal therapy ablation detection with medical diagnostic ultrasound. The endpoint of ablation is cell death or transition to another tissue state of health. The ablation is detected, but the therapy may continue for the same location or other locations. The system performs the method described above for FIG. 1 or a different method.

The ultrasound system includes a transmit beamformer 52, a transducer 54, a receive beamformer 56, an image processor 58, a display 60, a processor 62 and a memory 64. Other systems may be used. Additional, different or fewer components may be provided. For example, separate detectors and scan converter are also provided. As another example, a separate therapy transducer or treatment system is provided.

The detector of denaturation or cell death uses none, one or more input features from ultrasound data. Other sources of data include sensors, a therapy system, or other inputs. Such devices or inputs may be provided to the processor 62 or the memory 64. In one embodiment, all of the inputs features used by the detector are acquired from ultrasound data.

The system 10 is a medical diagnostic ultrasound imaging system. Imaging includes two-dimensional, three-dimensional, B-mode, Doppler, color flow, spectral Doppler, M-mode, strain, elasticity, or other imaging modalities now known or later developed. The ultrasound system 10 is a full size cart mounted system, a smaller portable system, a hand-held system or other now known or later developed ultrasound imaging system. In another embodiment, the processor 62 and memory 64 are part of a separate system. For example, the processor 62 and the memory 64 are a workstation or personal computer operating independently of or connected with the ultrasound system. As another example, the processor 62 and the memory 64 are part of a therapy system.

The transducer 54 is a single, one-dimensional, multi-dimensional or other now known or later developed ultrasound transducer. Each element of the transducer 54 is a piezoelectric, microelectromechanical, capacitive membrane ultrasound transducer, or other now known or later developed transduction element for converting between acoustic and electrical energy. Each of the transducer elements connect to the beamformers 52, 56 for receiving electrical energy from the transmit beamformer 52 and providing electrical energy responsive to acoustic echoes to the receive beamformer 56.

The transmit beamformer 12 is one or more waveform generators, amplifiers, delays, phase rotators, multipliers, summers, digital-to-analog converters, filters, combinations thereof, and other now known or later developed transmit beamformer components. The transmit beamformer 52 is configured into a plurality of channels for generating transmit signals for each element of a transmit aperture. The transmit signals for each element are delayed and apodized relative to each other for focusing acoustic energy along one or more scan lines. Signals of different amplitudes, frequencies, bandwidths, delays, spectral energy distributions or other characteristics are generated for one or more elements during a transmit event.

The receive beamformer 56 is configured to acquire ultrasound data representing a region of a patient. The ultrasound data is for measuring tissue related information, acquiring information, acquiring ultrasound data, and/or receiving other therapy data.

The receive beamformer 56 includes a plurality of channels for separately processing signals received from different elements of the transducer 54. Each channel may include delays, phase rotators, amplifiers, filters, multipliers, summers, analog-to-digital converters, control processors, combinations thereof and other now known or later developed receive beamformer components. The receive beamformer 56 also includes one or more summers for combining signals from different channels into a beamformed signal. A subsequent filter may also be provided. Other now known or later developed receive beamformers may be used. Electrical signals representing the acoustic echoes from a transmit event are passed to the channels of the receive beamformer 56. The receiver beamformer outputs in-phase and quadrature, radio frequency or other data representing one or more locations in a scanned region. The channel data or receive beamformed data prior to detection may be used by the processor 62.

The receive beamformed signals are subsequently detected and used to generate an ultrasound image by the image processor 58. The image processor 58 is a B-mode/M-mode detector, Doppler/flow/tissue motion estimator, harmonic detector, contrast agent detector, spectral Doppler estimator, combinations thereof, or other now known or later developed device for generating an image from received signals or ultrasound data. The image processor 58 may include a scan converter. The detected or estimated signals, prior to or after scan conversion, may be used by the processor 62.

The display 60 is a monitor, LCD, plasma, projector, printer, or other now known or later developed display device. The processor 62 and/or the image processor 58 generate display signals for the display 60. The display signals, such as RGB values, may be used by the processor 62.

The display 60 is configured to display an image representing the tissue during thermal therapy, such as a B-mode image of tissue. The display 60 may alternatively or additionally display an alert or indication of detection of the endpoint. The time at which the ablation occurred may be displayed. Alternatively, the ablation is indicated as a highlight of any locations where the endpoint has been reached. For example, the detector output is used to modulate the color of the B-mode image, such as showing locations with cell death as red. A % completion of ablation may be used. In other embodiments, the display 60 outputs an alert, such as a flashing warning indicating the occurrence of cell death. Audible warnings or instructions may be output.

The processor 62 is a control processor, beamformer processor, general processor, application specific integrated circuit, field programmable gate array, digital components, analog components, hardware circuit, combinations thereof and other now known or later developed devices for processing information.

The processor 62 is configured, with computer code, firmware, and/or hardware, to detect denaturation or cell death. The cell death in a region is detected. Locations distributed in a one-, two-, or three dimensional region are monitored. If or as cell death occurs at each of the locations, the processor 62 detects the event. The processor 62 implements a machine-learnt classier to detect the cell death. The classifier is a matrix, algorithm, or combinations thereof to estimate based on one or more input features.

The processor 62 receives, requests, and/or calculates values for the features input to the model. In one embodiment, one or more of the features and corresponding values are a function of the ultrasound data. The features from ultrasound data may undergo a transition at or just before cell death. The pattern of transition may be used to detect cell death.

Two or more different types of parameters are derived from ultrasound data. The parameters represent different characteristics of tissue response to ultrasound, such as decorrelation, displacement, and backscatter intensity. A single value is provided for each parameter per location. Multiple values per parameters may be applied to represent the feature at different times and/or locations. The parameter values of the input feature are from raw data, such as B-mode values, or are calculated, such as using tracking or correlation.

The processor 62 applies the values for a current time. The values are of current measures, previous measures, or changes between measures. In one embodiment, one or more of the features are previous outputs of the classifier. A time-dependent model is used. An initial input may be an assumed value, such as no cell death or a reference measurement before the start of therapy. The trend or change is accounted for by the feedback, allowing for predictive control of the thermal therapy. The feedback is of the raw output or is calculated from the previous output or outputs, such as a feature for adjacent locations having cell death.

In another embodiment, the processor 62 is configured to implement a temperature estimator. Using the same or different input features, the temperature estimator estimates the temperatures at various locations during thermal therapy. The temperature estimator may use one characteristic, such as speed of sound. Alternatively, the temperature estimator is a machine-learnt classifier that uses various ultrasound data and/or derived information to estimate the temperatures. The output of the temperature estimator may be used as an input feature for the detector of cell death and/or is used to display a temperature map for monitoring therapy.

The memory 64 is a computer readable storage medium having stored therein data representing instructions executable by the programmed processor for thermal therapy ablation detection with medical diagnostic ultrasound. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method of thermal therapy ablation detection with medical diagnostic ultrasound, the method comprising:
acquiring, with an ultrasound system, ultrasound data from a scan of tissue of a patient undergoing thermal therapy;
deriving, by a processor of the ultrasound system, information from the ultrasound data;
detecting, by the processor of the ultrasound system applying a classifier, a time point of death or transition towards death of the tissue based on input of the information;
outputting an indication of the time point.

2. The method of claim 1 wherein acquiring comprises acquiring the ultrasound data as B-mode data.

3. The method of claim 1 wherein deriving comprises calculating thermal strain and signal decorrelation.

4. The method of claim 3 wherein acquiring comprises acquiring the ultrasound data as B-mode data, and wherein detecting comprises detecting in response to the input comprising the strain, the signal decorrelation, and the B-mode data.

5. The method of claim 1 wherein deriving comprises deriving strain, displacement, backscatter power, signal decorrelation, shear wave velocity, elasticity, shear modulus, or combinations thereof.

6. The method of claim 1 wherein detecting comprises detecting with the classifier comprises a machine trained neural network, the information being input to the machine trained neural network and the machine trained neural network outputting the time point.

7. The method of claim 1 wherein detecting comprises inputting the information over time and the classifier detecting the time point.

8. The method of claim 1 wherein deriving comprises deriving different types of the information, and wherein detecting comprises detecting the time point from the different types of the information.

9. The method of claim 1 wherein detecting comprises detecting the time point of death or transition towards death of the tissue as a time of denaturation of the tissue.

10. The method of claim 1 wherein detecting the time point comprises detecting that the death of the tissue has occurred and where during the thermal therapy.

11. The method of claim 1 wherein outputting comprises outputting an image showing a location or locations where the time point of death has occurred.

12. The method of claim 1 wherein outputting comprises outputting the indication as an alert.

13. The method of claim 1 further comprising:
estimating temperatures as a function of location with another classifier responsive to the ultrasound data, the information, other ultrasound data, other information or combinations thereof;
triggering the detecting in response to one or more of the estimated temperatures by the other classifier.

14. The method of claim 13 wherein triggering comprises triggering when the one or more of the temperatures reaches a temperature threshold for imminent cell death.

15. In a non-transitory computer readable storage medium having stored therein data representing instructions executable by a programmed processor for thermal therapy ablation detection with medical diagnostic ultrasound, the storage medium comprising instructions for:
scanning, with a transducer, a patient with ultrasound during the thermal therapy;
calculating, with an ultrasound scanner, first and second types of tissue characteristics over time from response to the scanning;
identifying, by the processor and from the first and second types of tissue characteristics, a transition associated with denaturation of tissue; and
indicating the transition.

16. The non-transitory computer readable storage medium of claim 15 wherein identifying comprises identifying by the processor applying a machine learnt neural network.

17. The non-transitory computer readable storage medium of claim 15 wherein identifying comprises identifying a signature pattern of change of the first and second types of tissue associated with cell death of the tissue.

18. The non-transitory computer readable storage medium of claim 15 wherein calculating comprises calculating two or more of strain, displacement, backscatter power, correlation of signal, shear wave velocity, or elasticity.

19. The non-transitory computer readable storage medium of claim 15 further comprising monitoring temperature during the thermal therapy from the response to the scanning and switching to the identifying in response to the monitoring.

20. A system for thermal therapy ablation detection with medical diagnostic ultrasound, the system comprising:
a receive beamformer configured to acquire ultrasound data representing a region of a patient;
a processor configured to determine cell death in the region with a machine-trained classifier and an input feature vector of the machine-trained classifier comprising two or more types of parameters derived from the ultrasound data; and
a display configured to display an indication of the cell death.
